(19) United States
(12) Patent Application Publication
(10) Pub. No.: US 2022/0346906 A1
(43) Pub. Date: Nov. 3, 2022

(54) DEVICE TO PROMOTE BILATERAL SYMMETRY IN SURGICAL MARKINGS

(71) Applicant: Rona Z. Silkiss, Oakland, CA (US)

(72) Inventors: Rona Z. Silkiss, Oakland, CA (US); Maria Govorkova, Los Altos, CA (US)

(73) Assignee: Rona Z. Silkiss, Oakland, CA (US)

(21) Appl. No.: 17/695,903

(22) Filed: Mar. 16, 2022

Related U.S. Application Data
(60) Provisional application No. 63/258,389, filed on Apr. 28, 2021.

(51) Int. Cl. A61B 90/00

(52) U.S. Cl. CPC A61B 90/39; A61B 90/361; A61B 2090/3983; A61B 2090/3937; A61B 2090/3618; A61B 2090/366

(57) ABSTRACT

A device improves the outcome of bilateral eyelid, facial or other procedures, by improving the accuracy of bilateral pre surgical markings. The device may include a mirror, a camera, and a projector, where the projector projects an image captured by the camera onto a patient. Improved accuracy may be achieved using the device by projecting the mirror image of a pre-surgically marked reference eyelid or other body area onto the area of the other eyelid or contralateral body area that is similarly to be operated upon.

### BACKGROUND

One of the challenges of facial surgery is that small changes to facial appearance can be perceived both positively and negatively by patients who undergo the surgery. Although faces are not perfectly symmetric, enhanced symmetry has been associated with higher attractiveness. Humans are hard wired to recognize and appreciate symmetry, perhaps as a signal of good health. Even mild perturbations of symmetry may lead to an impression of unattractiveness or irregularity in facial appearance.

Blepharoplasty is a procedure in which an oculoplastic facial plastic or other specialty plastic surgeon reduces lax upper or lower eyelid skin and fat to improve eyelid function and/or cosmetic appearance. In blepharoplasty, promoting symmetrical outcomes are one of the most challenging aspects of bilateral surgery.

One of the common approaches in these surgeries is that one eyelid is first marked for surgery, the second eyelid then similarly marked. Obtaining the same crease, contour, and lateral tail after surgery between the right and left eyelid is critical to the postoperative result and patient satisfaction.

Current techniques of blepharoplasty surgery rely on caliper measurement and the “eye” of the surgeon marking the incisions, one eyelid at a time. This technique is time-consuming and invites the possibility of subjective views of the symmetry. There is a need for a precise, cost-effective, non-contact and simple surgical guide to improve pre surgical markings with resultant improvement in postoperative eyelid and other facial symmetry.

### SUMMARY OF THE EMBODIMENTS

This invention relates generally to a device for improving the outcome of bilateral eyelid or facial procedures by improving the accuracy of bilateral pre surgical markings, though the invention is applicable to any bilateral procedure requiring symmetry

In one embodiment the apparatus and method of this invention can be used in corrective/restorative eyelid surgery and more particularly to a device for capturing an image of a bilateral feature of a body part such as an eyelid and projecting the mirror image of that feature onto the corresponding contralateral location where the surgery is to be performed. In another embodiment, the invention covers a method for using the device of this invention in order to improve the symmetry, functionality and aesthetic outcomes of such surgery.

The invention includes a device that captures an image of the reference eyelid of a subject with its surgical markings, and then projecting the mirror image of that eyelid image/markings onto the other eyelid that is also to be surgically modified. The device includes a mirror for reflecting the first eyelid image and the mirror an orientation device to make fine adjustments in both the X and Y directions. The reflected image from the mirror may be captured by a camera, in one embodiment a digital camera. In another embodiment, the image from the mirror is first passed through a zoom objective where the size of the image can be scaled, the image from the camera then sent to a Pico projector where it can be projected onto the appropriate facial area of the subject. In one embodiment of the invention, depending upon the optics of the camera, a system can be provided to ensure the projected image is the mirror image of the reference eyelid.

A device for improving the accuracy of bilateral surgical markings includes a camera configured to capture image data of a first of two bilateral body parts; and a projector that projects projected image data captured by the camera onto a second of two bilateral body parts.

### BRIEF DESCRIPTION OF THE DRAWINGS

The present invention is described with respect to particular exemplary embodiments thereof and reference is accordingly made to the drawings in which:

FIG. 1 shows prior art surgical reference markings on a reference eye.

FIG. 2 is a schematic representation of the device of this invention, and its positioning during use.

FIG. 3 is a schematic representation of the device of this invention, and its positioning during use.

FIG. 4 shows the reference markings on the first eye compared to the overlay of the projected image and projected markings on the second eye.

FIG. 5 shows the device in use with a patient, it being understood that the device may be smaller than that shown, where the device has been shown larger than anticipated to show certain design elements.

FIG. 6 is a table that reports measured marking errors in percent.

FIG. 7 is a plot that compares the distribution of pixel-wise errors with and without the use of the device of this invention.

FIG. 8 is a visual guide to the significance of value of the median error.

FIG. 9 shows an angularly adjustable optics mount.

### DETAILED DESCRIPTION OF THE EMBODIMENTS

### Introduction

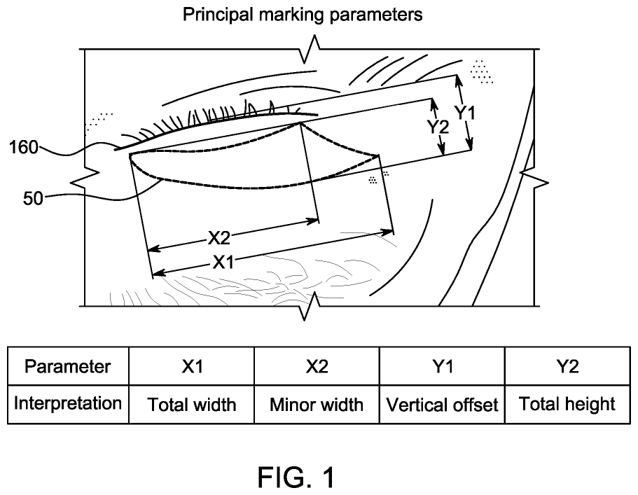

As shown in FIG. 1, a surgeon often marks one eye 160 with markings 50 (in the drawings, these markings are shown in phantom to differentiate them from other features of the eye, it being understood that the markings would most likely not be shown in phantom in practice, but as solid often colored lines). The markings noted within the dashed outline delineate tissue to be removed. The tissue inside the contour is removed and the wound is closed with a suture.

Device and Method

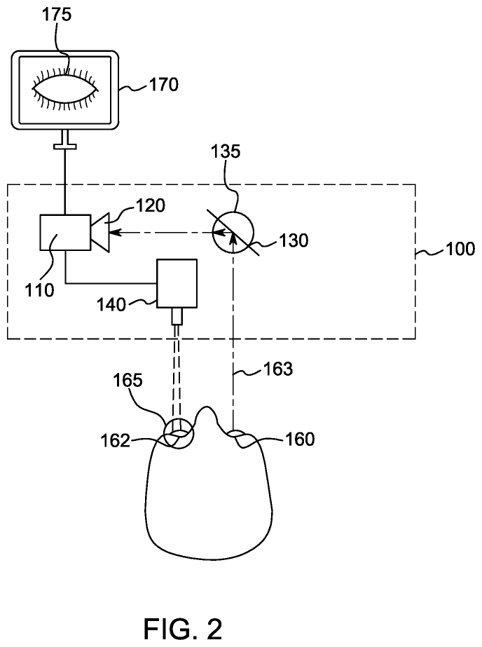

FIG. 2 shows the device 100 that includes a camera 110 with an optional zoom 120, a mirror 130, and a projector 140 that may be a pico projector. Each of these will be described in more detail in the description that follows.

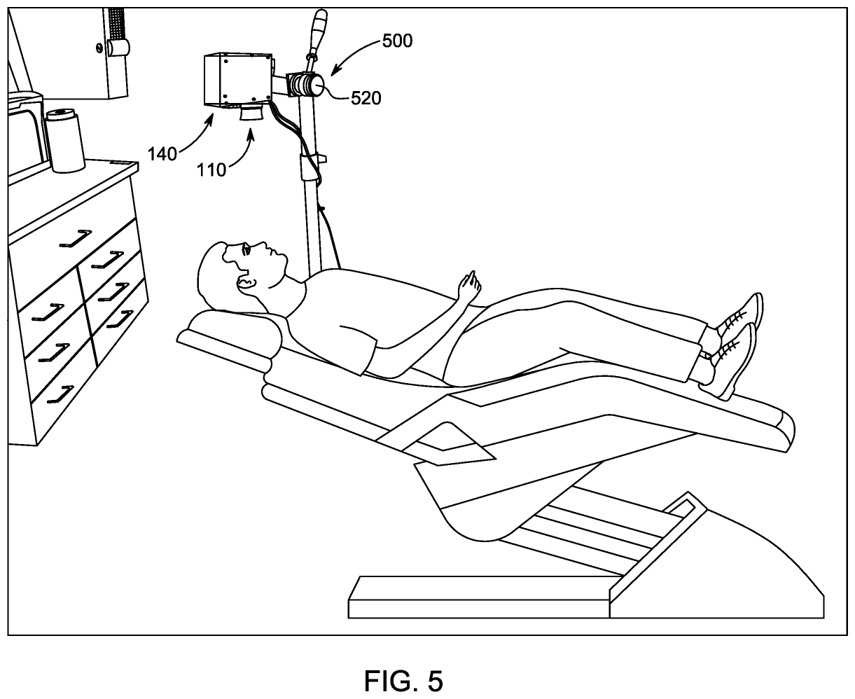

In use with a patent as shown in FIG. 5, using the device 100, a user/surgeon places certain reference markings 50 on a patient's first eye 160 in accordance with those shown in FIG. 1. These markings indicate which tissue should be removed by marking such tissue to be removed within the contour lines 50.

Then according to the device 100 shown in FIG. 2, the user aligns the mirror 130 to capture reflected light 163 from a patient's first reference eye (the right eye in FIGS. 1A and 1B) 160 and or aligns the camera 110 to capture the reflected light 163 on the mirror. 130. The user may confirm this alignment by watching a monitor projecting video 175 from the camera 110 or by looking at a projected image 165. The user aligns the projected image 165 onto the patient's second eye 132, where the projector 140 generates the projected image 165 using the video feed from the camera 110.

This projected image 165 from the reference eye includes the reference markings 50 captured from the reference eye 160 and the user positions the projector 140 to project those second markings appropriately over the second eye 162. The projected image data 165, it should be appreciated, will include the mirror image 167 of the reference markings 50 that were made on the first eye 160.

The user can then trace elements of the mirror image 167, including for example reference markings made on the first eye, onto the second eye 162 using an appropriate marking device and in this way, ensure a near perfect symmetry. This allows the reference features in the captured image (such as eyebrow, lid margin, eyelashes) to be overlaid precisely on the respective features of the second eye 162. FIG. 4 shows the reference markings 50 on the first eye 160 compared to the overlay of the projected image 165 and projected markings on the second eye 162.

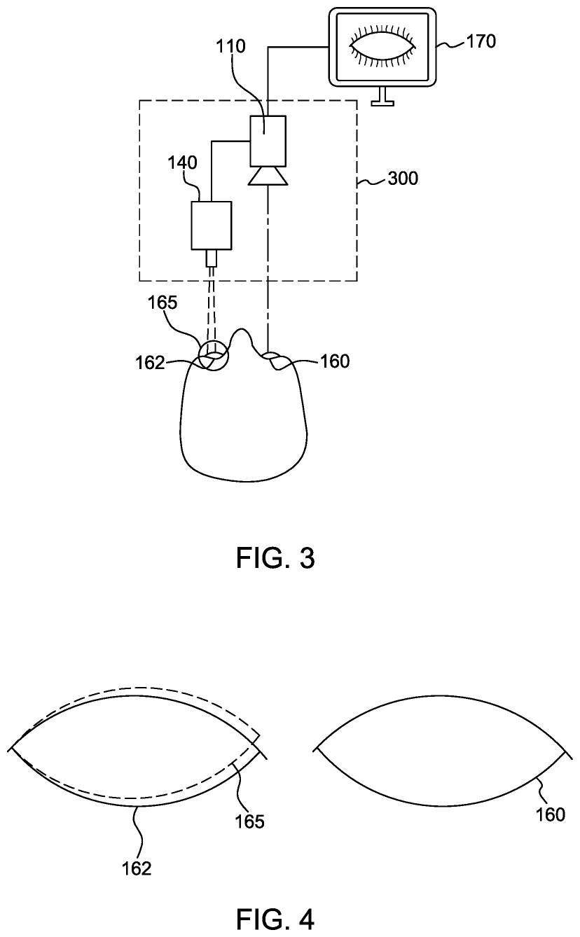

The mirror may not be part of the device 300 as shown in FIG. 3. In the embodiment of the device 300 in FIG. 3, the camera 110 may reverse the image of the first eye 160 or the projector 140 may reverse its output to generate the projected image data 165, without need for any mirror.

The mirror 130 may be mounted on an adjustable stand connected to or separate from the camera 110 and projector 140. This stand 500 allows for macro and micro adjustments in several directions and may include a rough adjustment mechanism 510 and a fine adjustment mechanism 520 of a type similar to the angularly adjustable optics Polaris Mirror Mount made by Thorlabs Inc. and shown in FIG. 9. The camera 110 and projector 140 may be similarly mounted.

The camera 110 may include optical wide/zoom 120 or digital zoom, or other image manipulation features like color filtering, wide angles, and effects. The camera may have multiple outputs and be wired or wirelessly connected to both the monitor 170 and/or the projector 110. The camera 110 and projector 140 may also be integral to one another. The camera 110 and projector 140 may be separate or integral to one another and yet move with respect to each other to allow for the camera 110 to align with the mirror 130 or reference eye 160 and the projector 140 to align with the second eye 162.

The camera 110 and/or projector 140 may include image stabilization software in order to ensure there is no shaking while projecting and tracing the reference markings 50.

The camera 110 may be a conventional CMOS video-camera, equipped with digital and/or optical zoom as mentioned.

The projector 140 may be a pico-projector and may be a consumer grade, low power miniature projector. It may project the projected image 165 using a Light Emitting Diode (LED). The projector 140 may include optical wide/zoom 120 or digital projection, or other image manipulation features like color filtering, wide angles, and effects. Any or all of these may be desirable according to the user.

In order to scale the image of the first marked eye, the device 100/300 may be periodically calibrated, i.e., to establish equivalence of scale of the image captured by the camera 110 and the projected image 165.

During calibration, a test pattern may be used for calibration. Here, an image of a first test pattern captured by the camera 110 is projected onto a second reference pattern that is similar to the test pattern. The purpose of calibration is to adjust the zoom ratio of the camera as well as the projection so that reference features of the two patterns overlap and the sizing of the captured image is identical to that projected.

The system shown may use a live video feed from the camera 120 to feed the projector 140. It may alternatively project a stored photo of the first eye 160 onto the second eye 162.

The device and method may be used in cosmetic and or reconstructive eyelid surgery. It should be understood, without departing from the scope or spirit of the invention that the device described herein may be used in other types of operations where bilateral symmetry is required such as mastopexy, breast augmentation, liposuction, facial rhytidectomy, abdominoplasty, etc. This device may also find utility in other, non-surgical applications such as symmetrical tattoo ink work.

Testing Results

The device was tested initially on a mannequin then on human volunteer subjects. No actual surgeries were performed. In the mannequin study, the direct measurements (with calipers) of the principal dimensions of the markings demonstrated statistically significant improvement of marking accuracy. For example, the random error of the total width of marking was improved from 8.1% to 2.0%. The error of height was improved from 12.1% to 7.4%. In the human subject study, eleven volunteer subjects were involved. All study data were anonymized. Subjects of all age groups, race or gender qualified for the study.

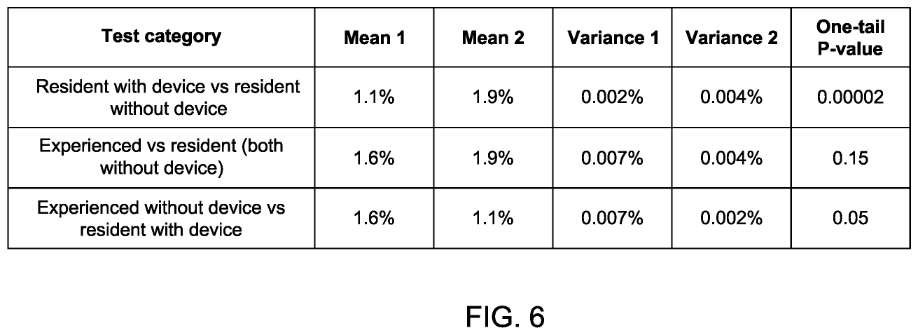

T-tests confirmed statistical significance of the differences between markings with and without device, as well as between experienced and novice resident surgeons (FIG. 6). For example, the resident with the device produced an integral marking error of 1.1% versus the error of 1.9% without the aid of the device (with resulting p-value of 0.00002). When comparing un-aided marking errors of the experienced surgeon with that of the resident, but aided by the device, the former was 1.6% versus 1.1%, with the p-value of 0.05.

While the average error in a percent range may not seem large, the nature of this integral metric is such that even quite significant but localized errors tend to average out in the final number.

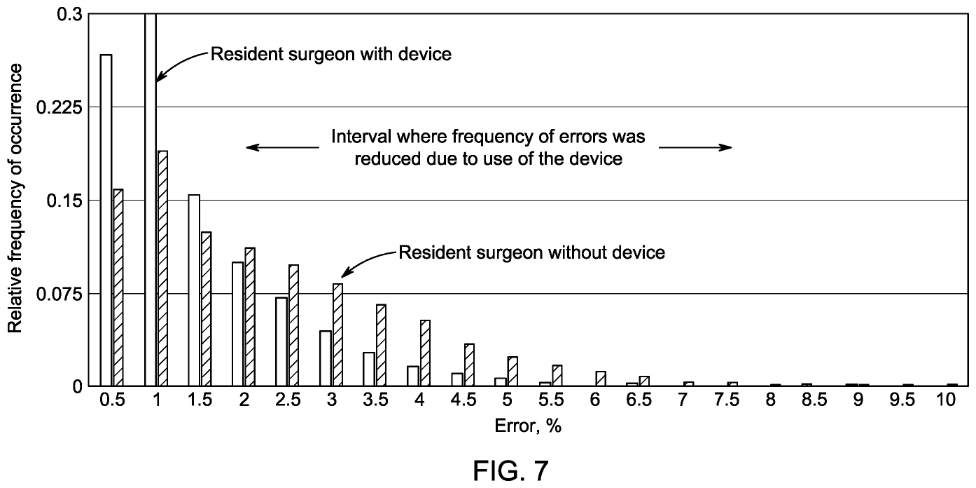

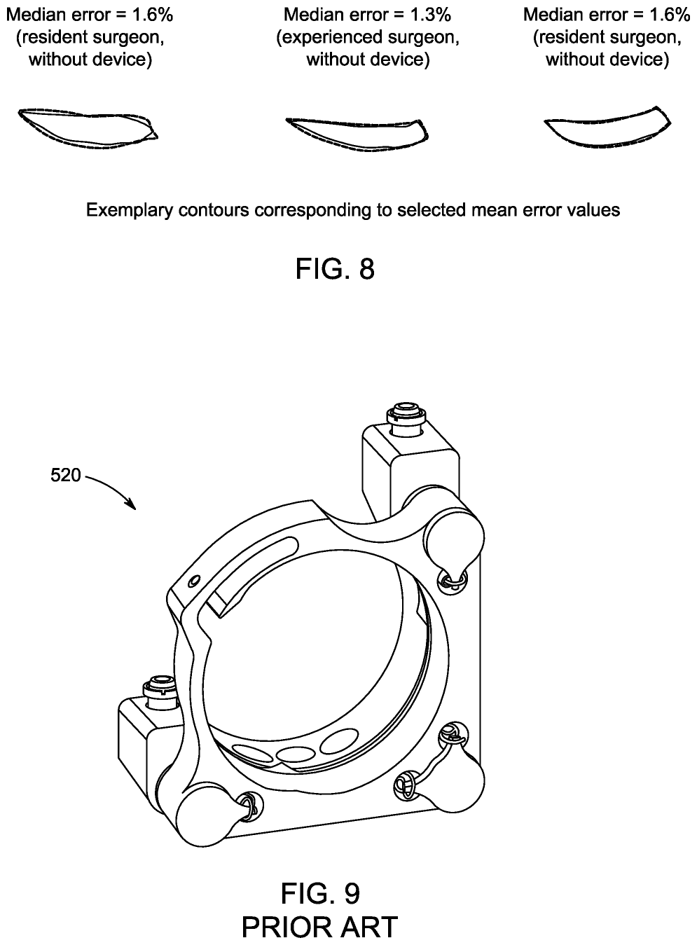

FIG. 8 includes representative images of contours corresponding to several mean error values. This demonstrates the significance of the differences. To further illustrate this, the distributions of the pixel-wise error values for each category of tests were calculated. The experienced surgeon versus the resident's distributions showed a clearly lower frequency of occurrence of larger errors in the interval from 2% to 6.5%. However, the resident aided with the device made fewer errors in the interval 2% to 7% when compared to unaided markings of the experienced surgeon (FIG. 7). Noted was that the pixel-wise errors shown are below roughly 2% are close to measurement error level and thus are insignificant. Noted also was the reduction of occurrence frequency of larger errors in the interval 2-7% when using the device.

In conclusion, surgical markings are placed on one eyelid then transferred to the contra lateral eyelid via the device. Once the drawings are completed and inspected for symmetry the surgical procedure begins by operating on one eyelid at a time in sequence. Markings made with the aid of the device of this invention were more precise. The device of this invention has a potential to greatly improve accuracy, reduce time and effort in producing symmetrical surgical markings. This is likely to improve symmetry, surgical outcome and patient satisfaction.

Surgical Method

1—Patient Prepping.

Under intravenous or general anesthesia, a 50:50 mixture of 2% xylocaine with 1:100,000 epinephrine, 0.5% Marcaine with 1;200,000 epinephrine, hyaluronidase and sodium bicarbonate or local anesthetic of the surgeon's choice may be injected into the area of the upper eyelids via the skin surface. The patient is then prepped and draped in the usual sterile fashion.

2—Device Calibration (to be Performed Periodically, e.g., Once a Day).

A test pattern (a paper or plastic stripe with a periodic and repeating grid pattern with equidistant markings) is placed under the device 100 at approximately the height of patient's eye. The image of the grid at the location of first eye is received by the camera 110 projected by the projector 140 onto a grid a location where the patient's second eye will be. The object here is to ensure that the pitch of grid lines is equal between the source test pattern and projected test pattern. To ensure this, both zoom and pointing of the device 100 are adjusted until the projected marks of the grid at the first eye location coincide with the marks on the strip at the second eye location.

3—Eyelid Marking

Attention is first directed to the upper eyelids where a marking pen and caliper are used to mark the intended eyelid incisions. These are carefully measured with a caliper and are drawn to specifications previously determined by the operative surgeon. This includes measurements of the natural lid fold crease, the amount of skin to be removed and its location, the amount of tissue to leave behind and its location. Care is taken to mark the lateral aspect of the incision so that it extends at a 45 degree or so angle in line with the cant of the lateral canthus and ultimately blends into a lateral canthal crow's foot crease. Once the surgeon is satisfied with the skin markings on the first eyelid, the device 100 is positioned as generally described already here. Several visual fiduciary markings are verified so that the projected markings from one eyelid are precisely positioned on the contralateral eyelid in a precisely symmetric location. This is achieved by adjusting the pointing of the projector 140. Note that the zoom value should not be adjusted at this time in order to preserve the calibration. Once positioned accurately into place, the projected marking is traced with a surgical pen onto the second eye 162.

4—Surgical Procedure

Then, the surgeon may proceed with the surgical procedure. A 5-0 silk is placed through the lid margin and the lid placed on downward tension. A #15 Bard Parker blade is used to the incise the skin directly in the markings previously measured and drawn. The cutting mode of cautery is used to excise skin. The orbital septum may be incised, and the fat trimmed according to the plan outlined prior to surgery. The incisions closed with 6-0 plain gut suture or other suture or glue of the operative surgeon's choice.

Attention is then directed to the contralateral eyelid, whereupon a #15 Bard Parker blade is used to incise the skin in the exact location of the previously traced markings. The procedure is completed as described for the opposite eyelid.

5—Postoperative Step

A combination corticosteroid antibiotic ointment applied. Ice packs applied.

### Embodiments

1. An embodiment including a device for improving the accuracy of bilateral surgical markings comprising:

a camera configured to capture image data of a first of two bilateral body parts; and

a projector that projects projected image data captured by the camera onto a second of two bilateral body parts.

2. The device of embodiment 1, wherein the projector displays a live video feed of the image data captured by the camera.

3. The device of embodiment 1, wherein the image data is a still image.

4. The device of embodiment 1, further comprising a mirror, wherein the image data captured by the camera is image data reflected from the first of two bilateral body parts onto the mirror.

5. The device of embodiment 1, wherein the camera includes a zoom feature that allows for zooming and or out.

6. The device of embodiment 5, wherein the zoom is digital.

7. The device of embodiment 5, wherein the zoom is optical.

8. The device of embodiment 1, wherein the projector includes a zoom feature that allows for projecting the image larger or smaller.

9. The device of embodiment 1, wherein the projected image data is manipulated using a filter.

10. The device of embodiment 1, wherein the projected image data is manipulated using effects.

11. An embodiment in a method of performing a bi-lateral surgery comprising:

marking a first of two bilateral body parts with surgical markings;

providing a camera configured to capture image data of the first of two bilateral body parts;

providing a projector that projects projected image data captured by the camera onto a second of two bilateral body parts;

aligning the projected image data over the second of two bilateral body parts;

marking the second of the two bilateral body parts with surgical markings based on the projected image data.

12. The method of embodiment 11, wherein the projector displays a live video feed of the image data captured by the camera.

13. The method of embodiment 11, wherein the image data is a still image.

14. The method of embodiment 11, further comprising a mirror, wherein the image data captured by the camera is image data reflected from the first of two bilateral body parts onto the mirror.

15. The method of embodiment 11, wherein the camera includes a zoom feature that allows for zooming and or out.

16. The method of embodiment 11, wherein the projector includes a zoom feature that allows for projecting the image larger or smaller.

17. The method of embodiment 11, wherein the projected image data is manipulated using a filter.

18. The method of embodiment 11, wherein the projected image data is manipulated using effects.

19. The method of embodiment 11, wherein the two bilateral body parts are eyes.

20. The method of embodiment 11, further comprising a step of calibrating the camera and the projector to ensure the projected image data projected onto the second of two bilateral body parts is accurate.

While the invention has been described with reference to the embodiments above, a person of ordinary skill in the art would understand that various changes or modifications may be made thereto without departing from the scope of the claims.

1. A device for improving the accuracy of bilateral surgical markings comprising:
a camera configured to capture image data of a first of two bilateral body parts; and
a projector that projects projected image data captured by the camera onto a second of two bilateral body parts.

2. The device of claim 1, wherein the projector displays a live video feed of the image data captured by the camera.

3. The device of claim 1, wherein the image data is a still image.

4. The device of claim 1, further comprising a mirror, wherein the image data captured by the camera is image data reflected from the first of two bilateral body parts onto the mirror.

5. The device of claim 1, wherein the camera includes a zoom feature that allows for zooming and or out.

6. The device of claim 5, wherein the zoom is digital.

7. The device of claim 5, wherein the zoom is optical.

8. The device of claim 1, wherein the projector includes a zoom feature that allows for projecting the image larger or smaller.

9. The device of claim 1, wherein the projected image data is manipulated using a filter.

10. The device of claim 1, wherein the projected image data is manipulated using effects.

11. A method of performing a bi-lateral surgery comprising:
marking a first of two bilateral body parts with surgical markings;
providing a camera configured to capture image data of the first of two bilateral body parts;
providing a projector that projects projected image data captured by the camera onto a second of two bilateral body parts;
aligning the projected image data over the second of two bilateral body parts;
marking the second of the two bilateral body parts with surgical markings based on the projected image data.

12. The method of claim 11, wherein the projector displays a live video feed of the image data captured by the camera.

13. The method of claim 11, wherein the image data is a still image.

14. The method of claim 11, further comprising a mirror, wherein the image data captured by the camera is image data reflected from the first of two bilateral body parts onto the mirror.

15. The method of claim 11, wherein the camera includes a zoom feature that allows for zooming and or out.

16. The method of claim 11, wherein the projector includes a zoom feature that allows for projecting the image larger or smaller.

17. The method of claim 11, wherein the projected image data is manipulated using a filter.

18. The method of claim 11, wherein the projected image data is manipulated using effects.

19. The method of claim 11, wherein the two bilateral body parts are eyes.

20. The method of claim 11, further comprising a step of calibrating the camera and the projector to ensure the projected image data projected onto the second of two bilateral body parts is accurate.
